(19) United States
(12) Patent Application Publication
(10) Pub. No.: US 2014/0255499 A1
(43) Pub. Date: Sep. 11, 2014

(54) Method of Forming Fine Particles of a Drug Substance

(71) Applicant: Jagotec AG, Muttenz (CH)

(72) Inventors: Guy Vergnault, Kembes (FR); Alain Nhamias, Bartenheim (FR); Pascal Grenier, Kappelen (FR)

(21) Appl. No.: 14/352,918

(22) PCT Filed: Oct. 5, 2012
(86) PCT No.: PCT/EP2012/069791

§ 371 Date: Apr. 18, 2014

(30) Foreign Application Priority Data
Oct. 21, 2011 (GB) 1118181.5

(51) Int. Cl. B01F 5/00; A61K 31/5415; A61K 31/216

(52) U.S. Cl. CPC B01F 5/008; A61K 31/216; A61K 31/5415
USPC 424/489; 366/176.3; 34/359; 514/545; 514/225.8

(57) ABSTRACT

A method of forming fine particles of a drug substance using a piston-gap high pressure homogeniser.

The invention is concerned with pharmaceutical compositions containing fine particles of a drug substance and to methods and materials for the preparation of said pharmaceutical compositions.

Many drug substances do not perform to their full potential because they have low bioavailability. This can result in an unfavourable therapeutic ratio (compromised efficacy and safety profile) caused by slow or variable drug uptake, increased side effects, poor patient compliance and higher dosing.

Reducing the particle size of a drug substance can improve its bioavailability and help overcome these disadvantages.

Studies have shown that by reducing the particle size of a drug it is possible to raise its absorption levels and increase delivery efficacy in the gastro-intestinal tract, as well as the vascular and pulmonary systems. As such, pharmaceutical compositions comprising fine particles of drug substances could be of particular benefit in the delivery of those substances having low solubility in aqueous media and/or low permeability.

Formulating drug substances as fine particles can also be of benefit in targeted drug delivery. For example, fine particles can be produced with dimensions that complement the vasculature of tumours and not that of healthy tissue. In this way, drug particles could be developed that are targeted to malign tissue only. Similarly, particles may be formed of sufficiently small diameter such that a drug substance can be administered systemically via the pulmonary route.

Further, fine particles can accumulate in certain organs by virtue of their particular surface properties. Accumulation of a compound in a target organ can reduce side effects and increase therapeutic efficiency.

Processes for forming fine particles have been described in the art. Such processes generally proceed by converting a coarse suspension of a drug substance consisting of particles with mean diameters in the micrometer range, and fracturing those particles to produce smaller particles.

One such method of forming fine particles involves the process of high pressure homogenisation. High pressure homogenisation is the process of breaking down particles suspended in a carrier fluid by forcing the fluid through a narrow gap in a valve, whereupon turbulence and impingement forces amongst other phenomena, cause the particles to break apart. A common type of apparatus for use in such homogenisation processes is a piston-gap homogeniser.

Piston gap homogenisers are well known in the art. Essentially, a piston gap homogeniser comprises a homogenisation valve through which a carrier fluid is expressed under pressure, whereupon particles entrained in the carrier fluid are fractured and homogenised. The valve typically comprises a valve body, housing a stationary valve seat in which a moveable piston is located. The piston is biased towards a closed position in which the piston abuts the valve seat in sealing contact. During the homogenisation process, however, a fluid is pumped into the valve body under a pressure which is sufficient to lift the piston slightly away from the valve seat. In such a spaced apart arrangement, opposing surfaces on the valves seat and the piston together define a narrow gap through which the carrier fluid is expressed and the particles homogenised. During the homogenisation process, it is desirable to control the pressure to maintain the gap as constant as possible. Poor pressure control can result in excessive oscillation of the valve piston into and out of contact with the valve seat. Excessive and repeated contacts can lead to wear on the valve surfaces, which is undesirable as it can lead to poor homogenisation, and the repair or replacement of valve components at frequent intervals, which can be costly.

Piston-gap homogenisers are commonly used to homogenise emulsions such as milk or orange juice. More recently they have also been employed to comminute particles of drug substances, as described in U.S. Pat. No. 5,858,410. However, despite all the advantages attendant with reducing the particle size of drug particles in this way, unlike the emulsion processes described above, for many drug substances it is often not possible to effect particle size reduction in a satisfactory manner. Various factors, including drug particle hardness or failure of the homogenising apparatus to reach and maintain sufficient pressure across the homogenisation valve, can result in failure to attain desired particle size reduction, or it may only be attained after prolonged processing time and after many cycles or passes through the valve. In certain other cases, particle size reduction may be achieved but the homogenisation conditions lead to degradation of the drug. During the homogenisation process, drug substances may be subjected to high mechanical and thermal stresses that may be excessive, particularly if the processing times are long, which can be the case if the drug particles are hard, as can be the case, for example, with crystalline drug substances.

There is a need to provide improved homogenisation processes. In particular, there is a need for processes to produce drug substances having an average particle size in the sub-micron (e.g. less than 1 micron) range, and more particularly, particles having an average particle size of less than 100 nm. Still more particularly, there is a need for improved homogenisation processes that can effectively comminute hard drug substances, such as crystalline drug substances. Still more particularly, there is a need for such homogenisation processes that can deliver desirable particle size reduction even in the case of drug substances that are chemically or physically labile such that they may be degradable under conventional homogenisation conditions. There is also a need to provide an improved homogenisation apparatus, the valve components of which, and particularly the piston, display an improved resistance to wear.

The present invention addresses problems of the prior art, and provides means for homogenising particles of a drug substance under conditions that promote particle size reduction but which are sufficiently gentle that there is no, or substantially no, degradation of the drug substance. In particular, the applicant has designed a homogenising valve suitable for use in a piston gap homogeniser to comminute drug substances, even drug substances that are hard, or drug substances that may be unstable and subject to degradation under prolonged exposure to thermal or mechanical stress, which valve is particularly resistant to wear.

According to the invention there is provided a method for the preparation of fine particles of a drug substance and to a method of forming a pharmaceutical composition containing said fine particles, the method comprises the step of comminuting a drug substance in a piston gap homogeniser comprising:

a body having a flow passage therethrough,
a homogenisation valve comprising first and second homogenising members disposed in said flow passage,
wherein each homogenising member has a homogenisation surface opposing the other, such that together the opposed surfaces define a gap that acts as a flow restriction in the flow passage and through which a coarse suspension of drug substance particles in a carrier fluid is expressed under pressure and discharged as a suspension of fine particles of drug substance, wherein at least one of the opposing homogenisation surfaces is arcuate.

In an embodiment of the invention the first homogenising member is in the form of a valve seat and the second homogenising member is in the form of a piston.

In an embodiment of the invention, the piston homogenisation surface opposing the valve seat is arcuate.

In an embodiment of the invention, the piston's homogenisation surface is a convex arcuate surface.

In an embodiment of the invention, the piston's homogenisation surface is spherical.

In an embodiment of the invention, the piston comprises a ball having a circumferential homogenisation surface that opposes the homogenisation surface on the valve seat.

In an embodiment of the invention, the valve seat is housed the body and is stationary, and the piston is moveable into and out of contact with the valve seat.

In an embodiment of the invention, the piston is biased towards and abuts the valve seat in sealed contact when the valve is not operational.

In an embodiment of the invention, when a coarse suspension is pumped into the valve under pressure the piston is urged away from the valve seat such that opposing homogenising surfaces on the valve seat and the piston are in spaced apart relationship and together define a gap through which the coarse suspension is expressed and discharged in the form of a fine suspension containing particles of a drug substance.

In an embodiment of the invention, the valve seat and piston are rotationally symmetrical and axially aligned such that when the coarse suspension is pumped through an axial bore in the valve seat under pressure, it is expressed radially though the gap defined by the piston and valve seat homogenising surfaces.

The fine particles produced by a method defined herein may be useful as a pharmaceutical dosage form as such, or they may be further processed into a pharmaceutical dosage form, as is more fully discussed below.

Accordingly, the invention provides in another aspect a method of forming a pharmaceutical dosage form comprising the step of forming fine particles of a drug substance according to the method defined herein.

The invention also provides in yet another of its aspects, a pharmaceutical dosage form formed by a method comprising the step of forming fine particles of a drug substance according to a method defined herein.

Further features and advantages of the invention will become apparent from the detailed description of specific embodiments of the invention, with reference to the appended drawings, which are provided purely as non-limiting examples. The drawings show only those details essential to an understanding of the present invention, and the positioning of the homogenizer valve in the homogenizer apparatus as well as its interaction with other components of said apparatus, which are both well known to persons skilled in the art, have been omitted.

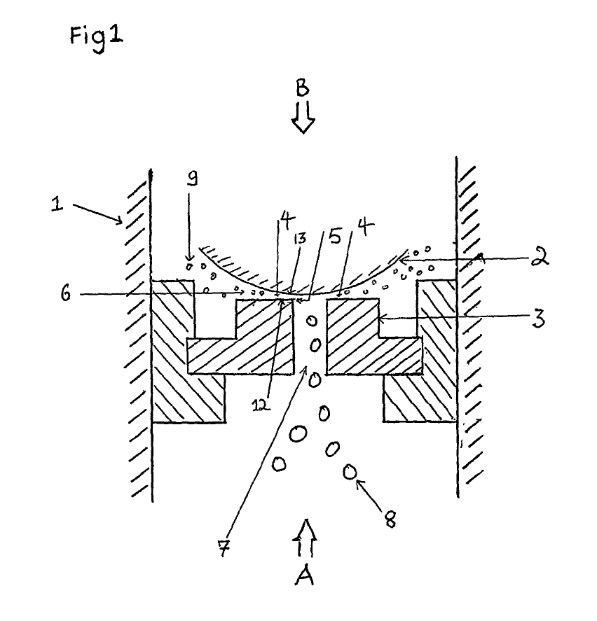

FIG. 1 represents a cross sectional view of a homogenisation valve

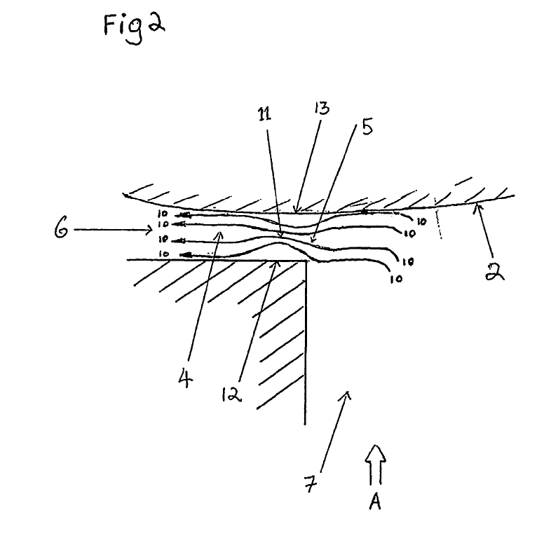

FIG. 2 is an expanded cross-sectional view of the homogenisation gap (4) shown in FIG. 1 in the region of the gap inlet (5)

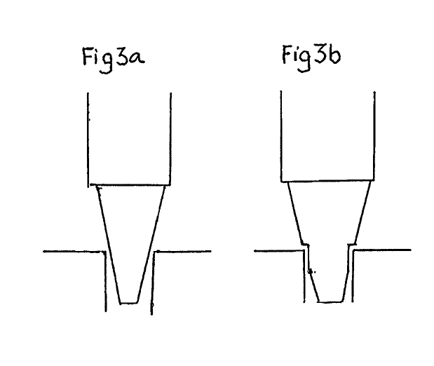

FIGS. 3 (a) and 3 (b) show a cross-sectional view of a valve comprising a cone-shaped piston. FIG. 3 (a) represents a view of the gap region when the valve piston is new, and FIG. 3 (b) represents a view of the same piston that is worn.

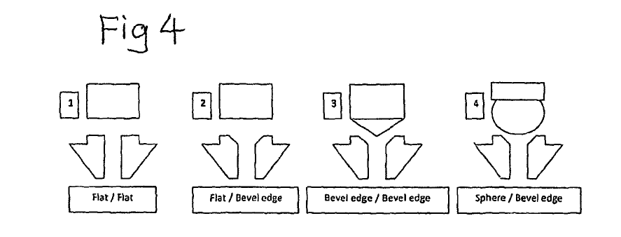

FIG. 4 is a series of schematic representations of valve designs

With reference to FIG. 1, a coarse suspension comprising particles of a drug substance (8) suspended in a carrier fluid is driven under pressure in a direction (A) by a feeder pump (not shown) towards a homogenisation valve (1) comprising a piston (2) (partly shown) and valve seat (3). The valve seat is stationary and is opposed by the piston, which is biased towards a closed position in which it is urged into contact with, and rests on, the valve seat. The biasing pressure in the direction (B) is applied to the piston typically by pneumatic or hydraulic means, although it might also be achieved in another construction by means of a grub screw, which acts via a spring, none of which biasing pressure means is shown in the drawings.

The valve in FIG. 1 is shown in its open position, whereby the piston (2) is spaced apart from the valve seat (3). This occurs when the coarse suspension pressure in the direction (A) is greater than the biasing pressure in the opposing direction (B). In this open position, the piston is in spaced apart relationship to the valve seat and the homogenising surface on the piston (13) and the homogenising surface on the valve seat (12), which opposes it together define a gap (4) having a gap inlet (5) and outlet (6). The gap width is defined by the opposing pressures in direction (A) and direction (B).

The valve seat comprises a central axial bore (7) through which flows a coarse suspension containing particles of drug substance (8) in a carrier fluid in the direction of the gap inlet (5). When the valve is in its open position the carrier fluid is forced through the gap inlet (5), whereupon the particles suspended in the fluid are broken down, and expressed as fine particles (9) from the outlet (6).

The gap (4) is very narrow. Typically, it might be between about 10 and 60 microns, more particularly about 25 to about 45 microns. The gap is maintained by the application of appropriate biasing pressure on the piston in the direction (B). The biasing pressure can be controlled to ensure the gap between valve seat and piston remains substantially constant even if there are pressure variations in the feeder pump during the homogenisation process. However, if the feeder pump pressure in the direction (A) drops below a certain level, the biasing pressure will be sufficient to close the piston on the valve seat. The valve will only re-open once the feeder pressure increases. Suitable pressure applied by the feeder pump may vary within wide ranges, for example from about 300 to 5000 bar. The homogenisation process may be carried out in a single step or it may be carried out in multiple steps. In particular, a coarse suspension may be subjected to a pre-homogenisation step at relatively low pressure, e.g. from about 350 to 700 bar, before being subjected to an homogenisation step at considerably higher pressure, e.g. from about 1300 to 1700 bar, or even higher.

This gap (4) is significantly narrower than the flow passage (7) and acts as an obstruction that restricts the flow of the coarse suspension. The restriction causes the suspension to be accelerated through the gap and as it does so the coarse particles (8) entrained in that suspension are subjected to impingement forces as they collide with each other and with the homogenising surfaces (13,12) of the piston and valve seat. These impingement forces and shear forces caused by the turbulent flow within the gap (4) play a role in the fracture and breakdown of the drug particles (8). At the same time as the suspension is accelerated, the static pressure on it drops suddenly and dramatically. This occurs because for non-compressible fluids, the sum of static and dynamic pressure is constant. So, given that when fluid velocity increases the dynamic pressure increases, it follows that static pressure must decrease. When the static pressure drops below the vapour pressure of the carrier fluid, the fluid boils. Finally, once the suspension exits the gap (4) through the outlet (6), the fluid decelerates and the static pressure on it increases. This sudden increase in static pressure causes the bubbles in the boiling fluid to implode violently. The implosion force further diminishes the dispersed particles in a process known as cavitation. In this manner, fine particles (9) are discharged through the outlet (6).

It is believed that cavitation is the major contributing factor in the comminution of drug particles. Accordingly, the greater the cavitation effect, the more efficient should be the particle size reduction process. The maximum cavitation effect is achieved when the pressure gradient between the region inside the gap (4) (low static pressure) and the region immediately outside the gap in the locality of the gap outlet (6) (high static pressure), is as large as possible. In order to keep a large pressure gradient, it is necessary to control the pressure inside the homogenisation gap (4). It is desirable to maintain the static pressure in the gap as low as possible.

FIG. 2 is a schematic representation of a view of the homogenisation gap (4) of the homogenisation valve shown in FIG. 1, in the region of the gap inlet (5).

The coarse suspension is fed from the central bore (7) in the valve seat towards the gap inlet (5). The inlet provides an obstruction in the flow path of the coarse suspension. As fluid streamlines (10) cannot abruptly change direction when they come upon an obstruction, and follow the contours of the piston and valve seat surfaces, they will initially maintain their direction and this will cause them to converge, albeit briefly and only slightly, immediately downstream of the obstruction, just inside the gap inlet (5).

The point of maximum convergence (and the minimum diameter of the carrier fluid stream) is referred to as the vena contracta (11). The vena contracta is situated in the gap immediately downstream of the obstruction provided by the gap inlet (5).

If the vena contracta is the point at which the fluid stream is at its narrowest, it is also at its highest velocity and here it reaches its lowest static pressure. Also, in the region of the vena contracta, the fluid stream is temporarily not in contact with the homogenising surfaces (13, 12) of the piston and valve seat respectively. As the fluid stream is not in communication with the homogenising surfaces at this point, the drag on the fluid is reduced, which promotes still further its acceleration.

Downstream of the vena contracta, the fluid streamlines start to diverge, and occupy the entire volume of the gap. At the same time, the fluid stream decelerates and experiences drag as it comes into communication with the homogenisation surfaces on the piston and valve seat. As the fluid stream decelerates, there is a concomitant increase in its static pressure. Ideally, this deceleration and increase in static pressure should be avoided to the greatest extent possible to ensure that the pressure gradient between the gap (4) and gap outlet (6) is as great as possible to achieve the highest cavitation effect.

As stated above, the gap between the opposing homgenisation surfaces is between 10 to 60 microns. The length of the vena contracta is approximately 14 times of this gap width, that is, between about 140 to 840 microns. In a particular embodiment of the invention, the gap length will be substantially similar in length as the vena contracta in order to maximise this pressure gradient, e.g. about 100 to about 1000 microns.

As already stated, placing an obstruction (in our case the gap inlet (5)) in a flow path, will create a vena contracta downstream of the obstruction. In principle, the more abrupt the obstruction, the more pronounced will be the vena contracta and the lower will be the static pressure in the homogenisation gap.

Accordingly, the applicant first employed a valve arrangement that was thought to provide the most abrupt obstruction to the flow of the coarse suspension. In particular, the applicant employed a valve seat that presented a square-edge at the region of the gap inlet, and a conical piston, the surface of which would meet the square-edge of the valve seat at a sharp angle to form the gap inlet. This arrangement is shown schematically in FIG. 3 (a). Such an arrangement of valve seat and piston is commonly employed to homogenise emulsion systems such as milk, orange juice and the like, and the applicant believed that this arrangement would have the advantage being simple, conventional and furthermore effective for the reason stated above. The applicant anticipated that this valve seat and piston arrangement would provide a gap inlet that presented the desired abrupt obstruction to the flow of fluid. However, when it was attempted to comminute drug particles, in particular hard drug particles such as crystalline particles, it was difficult to achieve and maintain a sufficiently high homogenisation pressure and obtain effective particle size reduction. Furthermore, process times increased and it was difficult to control of the temperature of the suspension. It was apparent that this arrangement of valve seat and piston was not optimal for comminuting particles of drug substances, particularly hard drug particles such as crystalline materials, and also drug substances that are somewhat sensitive to thermal degradation.

The reason for the poor performance became clear upon inspection of the valve piston: Extensive wear of the homogenising surface on the piston was observed, particularly in the form of groove, scratches or channels. When the piston is new, as shown in FIG. 3 (a), the area of contact between the piston and the valve seat in the closed position is very small, almost a single point of contact. Accordingly, when the piston is urged into a spaced apart relationship with respect to the valve seat to form the homogenisation gap, the homogenisation gap length is correspondingly very small. However, erosion of the piston surface by abrasive drug particles caused the gap length to effectively lengthen (see FIG. 3b). The increase in the effective gap length might, at least partly, explain the poor performance.

The applicant experimented with the shape of the conical piston by altering the angle of the cone from a very sharp angle to a much blunter angle, in order to examine whether this would have an effect on the efficiency of the process. Eventually, even a flat-faced piston was employed. In each case, practically no improvements in efficiency were observed.

The applicant also experimented with the shape of the valve seat, selecting a square-edged configuration at the gap inlet as shown in FIG. 1, and also various degrees of chamfer directed inwards in the direction of the gap inlet. The applicant found that the homogenisation efficiency did not appear to be particularly sensitive to the shape of the valve seat, however.

It was indeed surprising, therefore, that when a piston was employed having an arcuate homogenising surface opposing the valve seat at the gap inlet, it was possible to substantially improve homogenisation efficiency. It was also found that high homogenisation pressures could be attained and maintained with very little fluctuation. Application of consistently high pressures meant that drug substances could be fractured into fine particle rather more quickly than was possible with other valve arrangements. Relatively quick process times meant that temperature control of the suspension was not a problem and even thermally unstable drug substances could be homogenised without degradation. Still further, the piston displayed very little wear.

In a process according to the invention, it was possible to obtain suspensions of fine particles with a particle size distribution (D50), as measured by laser diffraction or photo-correlation spectroscopy of anything in the micron range, e.g 1 to 10 microns, or in the nanometre range, for example a D50 of 100 to 2000 nanometres. The particle size distribution (D50) will be to some extent drug-dependant, but for a given drug substance, the size reduction can be achieved in a comparatively short time. In view of the short process times, temperature is relatively straightforward to control and the partical size reduction can be achieved with little or no degradation of drug substance. The advantage of this process for the comminution of hard or thermally unstable drug substances is therefore apparent.

The advantage associated with the arcuate surface used in the homogenisation valve was surprising. For the reason given above, one would expect that a small gap length would promote cavitation and therefore particle size reduction. One would also expect that if the gap inlet presents an abrupt obstruction in the flow path of the coarse suspension this would promote the formation of a vena contracta. As such, it would seem that the valve seat and piston should be constructed as to present opposing surfaced with the smallest point of contact as possible at the gap inlet, as well as to present the most abrupt obstruction to the flow path at the gap inlet.

The arcuate homogenisation surface opposing the valve seat at the gap inlet would not seem to be the optimal way to achieve this, because the curvature of the surface at the gap inlet would not present the most abrupt obstruction to the flow path, nor would an arcuate surface appear to oppose the valve seat at the gap inlet with the smallest point of contact possible. Yet, the valve comprising the arcuate surface provided very efficient and reproducible particle size reduction.

Without being bound by any theory, it would seem that the arcuate surface was particularly resistant to wear. In view of the relative lack of wear, it is possible to obtain high homogenisation pressure and maintain it. Consistent application of high pressure enables the particle reduction process to proceed in a relatively short period of time. Accordingly, whereas other valve seat and piston designs should theoretically perform better than the arcuate piston, due to rapid wear it appears it is not possible to apply consistently high homogenisation pressures.

Given that drug particles could be broken down into fine particles in a relatively short space of time, the method of the present invention can be particularly useful to comminute particles of drug substances that are susceptible to thermal degradation.

The arcuate piston surface is most conveniently provided in the form of a ball bearing that is attached, more particularly releasably attached, to the end of the body of a piston that is adapted to receive the ball. The advantage of this construction is its relative simplicity, such that if the ball is eventually worn, it can be replaced inexpensively rather than replacing or reconditioning an entire piston.

The ball can be attached to the piston by any manner of suitable means. It can be welded or glued to the piston, or it can be mechanically fixed, such as by means of a lug formed in the piston that is adapted to fit into a hole formed in the ball, or it can be a combination of any of these means.

Another possible fixing means is to fix the ball into a socket formed in the piston. The socket may be configured such that the ball is held securely in place but is allowed to rotate in the socket in response to fluid moving across its surface. This arrangement can be particularly advantageous as the rotation ensures that the entire spherical surface of the ball successively forms the homogenisation surface of the piston that is proximate to and opposing the valve seat. In this way, there is much reduced wear to the piston homogenisation surface and efficient homogenisation can be maintained over a longer serviceable life time.

As stated hereinabove, the valve seat and the piston are typically both substantially rotationally symmetric and co-axially aligned. As such, when the piston homogenisation surface is in contact with the valve seat, together they form an annular seal. When they are in spaced apart relationship they define an annular gap, through which the coarse suspension will be expressed radially. The radial path represents the shortest path through the annular gap. Any deviation from this radial path will effectively increase the gap length, reduce the cavitation force and affect homogenisation efficiency.

Fissures, cracks or scratches on either or both the opposing homogenising surfaces on the valve seat and piston may create alternative flow paths for the carrier fluid, deviating it from the shortest (i.e. radial) pathway through the homogenisation gap and adversely affecting the process of cavitation. Accordingly, the homogenisation surfaces should be highly polished and as free from imperfections as possible. Furthermore, because abrasive drug particles may cause scratches during the process of homogenisation, the piston and valve seat should be checked regularly and reconditioned or replaced as necessary.

It is inconvenient, time-consuming and costly to have to either replace the piston or polish it at regular intervals. One of the advantages of using a ball releasably attached to a piston is that if replacement is required, it is necessary only to replace the relatively inexpensive ball rather than replace a complete piston or recondition it for further use. Balls are relatively inexpensive to replace and it is preferred to use such balls as part of the piston. The balls can, of course, be fabricated from any material that is hard and sufficiently robust to resist abrasive forces. Suitable materials include metals and ceramics materials.

The roughness of the homogenisation surface on the piston and on the valve seat should be as low as possible. Roughness is a measure of the texture of a surface. It is quantified by the vertical deviations of a real surface from its ideal form. If these deviations are large, the surface is rough; if they are small the surface is smooth.

Roughness plays an important role in determining how a coarse suspension will flow through the homogenising gap. Rough surfaces have higher friction coefficients than smooth surfaces. Irregularities in the surface may form alternative pathways for fluid flow and also increase drag, slowing down the speed at which the fluid passes through the gap. Roughness is undesirable for these reasons. It can adversely affect performance of the homogenisation process and can be difficult and expensive to control. However, decreasing the roughness of a surface, can increase manufacturing and running costs. The piston with an arcuate homogenising surface, and in particular the use of a ball ensures desirable combination of low cost and good performance.

Surface roughness can be reduced using many techniques such as grinding, polishing, electro-polishing and the like. Preferably, surface roughness should be in the micron to sub-micron range, e.g. 50 to 0.02 microns, more particularly 5 to 0.02 microns, still more particularly 1 to 0.2 microns or less. Most preferably the surface roughness is reduced by polishing to a mirror state

Surface roughness can be measured by techniques known in the art. Any calibrated means of determining the peaks and valleys of the surface being evaluated can be used. Non-contact equipment such as lasers can be employed, as well as various contact tools that are available, which ride over the peaks and troughs on the surface.

The homogeniser device and methods described hereinabove are suitable for homogenising drug particles to produce fine suspensions of drug particles.

The fine suspensions emerging from the homogenising valve can be analyses for particle size distribution. If the particle size distribution is not as desired, the fine suspension can be reprocessed through the valve via a recycle loop in the homogenisation device.

The particle size refers to the median dimension of particles in a sample and it may be based on the number of particles, the volume of the particles, or the mass of the particles. The particle size may be obtained using any of a number of standard measuring techniques including laser diffraction methods, or photo correlation spectroscopy. Unless stated to the contrary, all particle sizes mentioned in this specification relate to the median particle size based on volume.

The median particle size (diameter) of the particles in a fine suspension produced according to a method of the present invention may vary between about 10 nm and 5 microns, more particularly 100 to 1,000 nm (determined by photon correlation spectroscopy), the distribution of the population being quite narrow.

Once the desired particle size is achieved, the fine suspension can be collected and stored for later use, or fed directly into formulating apparatus, for example a mixer or granulator, for further processing into finished pharmaceutical compositions. For example, a fine suspension may be lyophilized or spray dried, or incorporated into a solid carrier matrix, according to methods well known in the art and which will be described in more detail below.

The fields of use for the pharmaceutical compositions according to the invention are diverse and the fine suspensions may be formulated accordingly. For example, they can be employed in oral, parenteral, topical or inhalable dosage forms.

All manner of drug substances can be processed in a method according to the present invention. Reducing the particle size of a drug substance effectively increases its surface area and may increase its solubility. For this reason, the process of the present invention is suitable for formulating poorly soluble drug substances.

Fine particles of drug substances can also be useful in targeted drug delivery. For example, fine particles can be produced with dimensions that complement the vasculature of tumours and not that of healthy tissue. In this way, drug particles could be developed that are targeted to malign tissue only. Similarly, particles may be formed of sufficiently small diameter such that a drug substance can be administered systemically via the pulmonary route.

Accordingly, with prejudice to the generality of application of the present invention, the method may be particularly suitable for preparing fine particles of anti-cancer compounds or compounds for delivery by the pulmonary route.

Still further, as the method according to the present invention performs particle size reduction efficiently, it is particularly suitable for comminuting such compounds that are rather hard, for example drug substances existing in one or more crystalline phase, or of drug substances that are susceptible to degradation or polymorphic change as a result of thermal stress.

By “poorly soluble” is meant those compounds that include those that are classified either as “sparingly soluble”, “slightly soluble”, “very slightly soluble” or “practically insoluble” in the United States Pharmacopoeia, i.e. compounds having a solubility of one part of solute to about 30 to about 100 parts of solvent, about 100 to 1000 parts solvent; about 1000 to about 10000 parts of solvent; or about 10000 parts or greater of solvent, when measured at room temperature, e.g. about 17 to 25 degrees centigrade and a pH of about 2 to 12.

Non-limiting examples of some drugs that may be employed in the present invention include immunosuppressive and immunoactive agents, antiviral and antifungal agents, antineoplastic agents, analgesic and antiinflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic and cholinomimetic agents, antimuscarinic and muscarinic agents, antiadrenergic and antarrhythmics, antihypertensive agents, antineoplastic agents, hormones, nutrients, anesthetic agents, ace inhibiting agents, antithrombotic agents, anti-allergic agents, antibacterial agents, antibiotic agents, anticoagulant agents, anticancer agents, antidiabetic agents, antihypertension agents, antifungal agents, antihypotensive agents, antiinflammatory agents, antimicotic agents, antimigraine agents, antiparkinson agents, antirheumatic agents, antithrombins, antiviral agents, beta blockering agents, bronchospamolytic agents, calcium antagonists, cardiovascular agents, cardiac glycosidic agents, carotenoids, cephalosporins, contraceptive agents, cytostatic agents, diuretic agents, enkephalins, fibrinolytic agents, growth hormones, immunosurpressants, insulins, interferons, lactation inhibiting agents, lipid-lowering agents, lymphokines, neurologic agents, prostacyclins, prostaglandins, psycho-pharmaceutical agents, protease inhibitors, magnetic resonance diagnostic imaging agents, reproductive control hormones, sedative agents, sex hormones, somatostatins, steroid hormonal agents, vaccines, vasodilating agents, and vitamins.

A detailed description of these and other suitable drugs may be found in Remington's Pharmaceutical Sciences, 18th edition, 1990, Mack Publishing Co. Philadelphia, Pa. which is hereby incorporated by reference.

Non-limiting examples of representative poorly soluble drugs can be selected from the group consisting albendazole, albendazole sulfoxide, alfaxalone, acetyl digoxin, acyclovir analogs, alprostadil, aminofostin, anipamil, antithrombin matenolol, azidothymidine, beclobrate, beclomethasone, belomycin, benzocaine and derivatives, beta carotene, beta endorphin, beta interferon, bezafibrate, binovum, biperiden, bromazepam, bromocryptine, bucindolol, buflomedil, bupivacaine, busulfan, cadralazine, camptothesin, canthaxanthin, captopril, carbamazepine, carboprost, cefalexin, cefalotin, cefamandole, cefazedone, cefluoroxime, cefmenoxime, cefoperazone, cefotaxime, cefoxitin, cefsulodin, ceftizoxime, chlorambucil, chromoglycinic acid, ciclonicate, ciglitazone, clonidine, cortexolone, corticosterone, cortisol, cortisone, cyclophosphamide, cyclosporin A and other cyclosporins, cytarabine, desocryptin, desogestrel, dexamethasone esters such as the acetate, dezocine, diazepam, diclofenac, dideoxyadenosine, dideoxyinosine, digitoxin, digoxin, dihydroergotamine, dihydroergotoxin, diltiazem, dopamine antagonists, doxorubicin, econazole, endralazine, enkephalin, enalapril, epoprostenol, estradiol, estramustine, etofibrate, etoposide, factor ix, factor viii, felbamate, fenbendazole, fenofibrate, flunarizin, flurbiprofen, 5-fluorouracil, flurazepam, fosfomycin, fosmidomycin, furosemide, gallopamil, gamma interferon, gentamicin, gepefrine, gliclazide, glipizide, griseofulvin, haptoglobulin, hepatitis B vaccine, hydralazine, hydrochlorothiazide, hydrocortisone, ibuprofen, ibuproxam, indinavir, indomethacin, iodinated aromatic x-ray contrast agents such as iodamide, ipratropium bromide, ketoconazole, ketoprofen, ketotifen, ketotifen fumarate, K-strophanthin, labetalol, lactobacillus vaccine, lidocaine, lidoflazin, lisuride, lisuride hydrogen maleate, lorazepam, lovastatin, mefenamic acid, melphalan, memantin, mesulergin, metergoline, methotrexate, methyl digoxin, methylprednisolone, metronidazole, metisoprenol, metipranolol, metkephamide, metolazone, metoprolol, metoprolol tartrate, miconazole, miconazole nitrate, minoxidil, misonidazol, molsidomin, nadolol, nafiverine, nafazatrom, naproxen, natural insulins, nesapidil, nicardipine, nicorandil, nifedipine, niludipin, nimodipine, nitrazepam, nitrendipine, nitrocamptothesin, 9-nitrocamptothesin, oxazepam, oxprenolol, oxytetracycline, penicillins such as penicillin G benethamine, penecillin O, phenylbutazone, picotamide, pindolol, piposulfan, piretanide, piribedil, piroxicam, pirprofen, plasminogenic activator, prednisolone, prednisone, pregnenolone, procarbacin, procaterol, progesterone, proinsulin, propafenone, propanolol, propentofyllin, propofol, propranolol, rifapentin, simvastatin, semi-synthetic insulins, sobrerol, somastotine and its derivatives, somatropin, stilamine, sulfinalol hydrochloride, sulfinpyrazone, suloctidil, suprofen, sulproston, synthetic insulins, talinolol, taxol, taxotere, testosterone, testosterone propionate, testosterone undecanoate, tetracane, tiaramide HCl, tolmetin, tranilast, triquilar, tromantadine HCl, urokinase, valium, verapamil, vidarabine, vidarabine phosphate sodium salt, vinblastine, vinburin, vincamine, vincristine, vindesine, vinpocetine, vitamin A, vitamin E succinate and x-ray contrast agents.

Drugs can be a neutral species or basic or acidic as well as salts such as exist in the presence of an aqueous buffer.

Drug concentration in the coarse suspension can affect the efficiency of the particle size reduction process, in that the amount of drug suspended will influence the viscosity of the coarse suspension. In a particular embodiment of the present invention, the amount of drug employed may range between about 5% to about 20% by weight, more particularly about 7 to 17% by weight, e.g. about 10% by weight.

Viscosity of the coarse suspension may range in value up to about 1 mPa·s, still more particularly about 10⁻³ mPa·s (1 cps) to about 5.10⁻² mPa·s (50 cps).

In addition to the drug substance and the liquid carrier in which it is suspended, the solid and liquid components of the coarse suspension processed according to a method of the present invention may include processing and dispersing aids (surfactants and stabilizers) and other excipients found in pharmaceutical dosage forms. These excipients may include, without limitation, low melting ethylene oxides (PEGs); oils, such as arachis oil, cottonseed oil, sunflower oil, and the like; semisolid lipophilic vehicles, such as hydrogenated specialty oils, cetyl alcohol, stearyl alcohol, gelucires, glyceryl behenate, and the like; solubilizing or emulsifying agents, such as Tween 80, SLS, CTAB, sodium deoxycholate, Imwitor, Cremophor, Poloxamer, and the like; and surface stabilizers, including cetyl pyridinium chloride, gelatin, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, sodium dodecylsulfate, carboxymethylcellulose calcium, hydroxypropyl celluloses, hydroxypropyl methylcellulose, hydroxypropylcellulose, carboxymethylcellulose sodium, methylcellulose, hydroxyethylcellulose, hydroxypropylmethyl-cellulose phthalate, noncrystalline cellulose, polyvinyl alcohol, polyvinylpyrrolidone, 4-(1,1,3,3-tetramethylbutyl)-phenol polymer with ethylene oxide and formaldehyde, poloxamers, poloxamines, dimyristoyl phophatidyl glycerol, dioctylsulfosuccinate, diallcylesters of sodium sulfosuccinic acid, sodium lauryl sulfate, an alkyl aryl polyether sulfonate, a mixture of sucrose stearate and sucrose distearate, p-isononylphenoxypoly-(glycidol), block copolymers of ethylene oxide and propylene oxide, and triblock copolymers and having a molecular weight (number average) of about 5000, and the like. Many of these surface stabilizers are known pharmaceutical excipients and are described in the Handbook of Pharmaceutical Excipients, published jointly by the American Pharmaceutical Association and The Pharmaceutical Society of Great Britain (1986), which is herein incorporated by reference. The surface stabilizers are commercially available or may be prepared by known techniques.

In a particular embodiment of the present invention, the surfactant is non.ionic. Still more particularly, the surfactant may be an inhibitor of the efflux pump transporter protein Pgp or P-glycoprotein. Representative of these surfactants are fatty acid ester surfactants comprising a polyoxyethylene moiety, such as:

polyethoxylated tocopheryl succinate (TPGS), polyoxyethylene castor oil or polyethoxylated castor oil (Crémophor® EL), polyoxyethylene sorbitan monolaurate (Tween® 20), polyoxyethylene sorbitan monopalmitate (Tween® 40), polyoxyethylene sorbitan monostearate (Tween 60), polyoxyethylene sorbitan monooleate (Tween® 80), polyoxyethylene-polyoxypropylenele|copolymers (Pluronic® P85 et L81), octylphenolethoxylate (Triton X 100) and Nonylphenyl polyoxyethyleneglycol (Nonidet P40).

In a more particular embodiment of the present invention the surfactant is vitamin E TPGS.

The coarse suspension generally includes about 0.01 to about 10% w/w of one or more surfactants, and often includes about 0.1 to about 3% w/w of surfactants.

In addition, the coarse suspension generally includes about 0 to about 30% w/w of one or more surface stabilizers and often includes about 0 to about 12% w/w of surface stabilizers. In many cases, the coarse suspension includes about 0 to about 8% w/w of surface stabilizers.

Further processing of the fine suspensions formed according to a method of the present invention may comprise feeding of the suspension into a high-shear, wet granulator. One or more feeders supply the wet granulator with pharmaceutically acceptable excipients, which help stabilize the suspension. The resulting wet granulation of stabilized fine particles may enter a dryer e.g., a convective heat dryer, such as a fluid bed dryer, a radiant heat dryer, such as an IR tunnel dryer, and the like, which removes any residual liquid.

Useful high-shear, wet granulators include, without limitation, twin-screw mixers, planetary mixers, high-speed mixers, extruder-spheronizers and the like. Other useful wet granulators include fluidized bed granulators. Like spray drying, fluidized bed granulation is a low-shear granulation method. However, as its name suggests, fluidized bed granulation involves spray-coating a fluidized bed of particles containing excipients, with a liquid fine suspension of drug substance. In contrast, spray drying involves spraying a drug substance fine suspension into a hot gas in order to produce granules; the suspension comprises discrete particles of drug substance dispersed in a liquid carrier, as well as one or more excipients, which are dissolved in the liquid carrier. For a discussion of useful wet granulators, see M. Summers & M. Aulton, Dosage Form Design and Manufacture 25:364-78 (2d ed., 2001), the complete disclosure of which is herein incorporated by reference.

Alternatively, the fine suspension exiting the homogenizer may be combined in a low-shear mixer or blender with one or more pharmaceutically acceptable excipients, which the system supplies through one or more feeders. The excipients help stabilize the fine suspension. The resulting slurry from the blender may enter a spray dryer, which drives off the liquid carrier and produces a dry granulation of fine particles and excipients.

Useful excipients include, without limitation, lactose, mannitol, sorbitol, sucrose, trehalose, xylitol, dextrates, dextran, dextrose, and the like. The amounts of any excipients added during granulation will depend on the desired drug loading in the dry granulation. In most cases, the drug substance comprises from about 5% w/w to about 95% w/w of the dry granulation and often comprises from about 5% w/w to about 65% w/w of the dry granulation. For a discussion of useful excipients that may be used to stabilize the fine suspension, see U.S. Pat. No. 5,571,536 and U.S. Pat. No. 6,153,225, which are herein incorporated by reference in their entirety.

The resulting dry granulation (which may have an average particle size of about 250 microns to about 2000 microns may) be stored, used to make a pharmaceutical composition, or directly fed to an optional milling operation, where the size of the granulate is reduced. Useful milling equipment includes jet mills (dry), ball mills, hammer mills, and the like. The milled granulation is combined with additional pharmaceutically acceptable excipients, if necessary, from one or more solids feeders. The resulting mixture undergoes dry blending to form a drug product, which may optionally undergo further operations, such as tableting or encapsulation, coating, and the like, to form the final pharmaceutical composition. For a discussion of drying, milling, dry blending, tableting, encapsulation, coating, and the like, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997), which are herein incorporated by reference.

For pharmaceutical compositions in tablet form, depending on dose, the drug may comprise about 1% to about 80% of the pharmaceutical composition, but more typically comprises about 5% to about 65% of the composition, based on weight. In addition to the drug substance, the tablets may include one or more disintegrants, surfactants, glidants, lubricants, binding agents, and diluents, either alone or in combination. Examples of disintegrants include, without limitation, sodium starch glycolate; carboxymethylcellulose, including its sodium and calcium salts; croscarmellose; crospovidone, including its sodium salt; PVP, methylcellulose; microcrystalline cellulose; one- to six-carbon alkyl-substituted HPC; starch; pregelatinized starch; sodium alginate; and mixtures thereof. The disintegrant will generally comprise about 1% to about 25% of the pharmaceutical composition, or more typically, about 5% to about 20% of the composition, based on weight.

Tablets may optionally include surfactants, such as SLS and polysorbate 80; glidants, such as silicon dioxide and talc; and lubricants, such as magnesium stearate, calcium stearate, zinc stearate, sodium stearyl fumarate, sodium lauryl sulfate, and mixtures thereof. When present, surfactants may comprise about 0.2% to about 5% of the tablet; glidants may comprise about 0.2% to about 1% of the tablet; and lubricants may comprise about 0.25% to about 10%, or more typically, about 0.5% to about 3% of the tablet, based on weight.

As noted above, tablet formulations may include binders and diluents. Binders are generally used to impart cohesive qualities to the tablet formulation and typically comprise about 10% or more of the tablet based on weight. Examples of binders include, without limitation, microcrystalline cellulose, gelatin, sugars, polyethylene glycol, natural and synthetic gums, PVP, pre-gelatinized starch, HPC, and HPMC. One or more diluents may make up the balance of the tablet formulation. Examples of diluents include, without limitation, lactose monohydrate, spray-dried lactose monohydrate, anhydrous lactose, and the like; mannitol; xylitol; dextrose; sucrose; sorbitol; microcrystalline cellulose; starch; dibasic calcium phosphate dihydrate; and mixtures thereof.

The invention is illustrated in further detail in the following examples.

### EXAMPLE 1

### Piston/Valve Design Evolution (Fenofibrate)

The following experiment was designed to show the effect of the valve design on the homogenisation of a fenofibrate suspension.

The homogenisation equipment employed was an Emulsiflex C50 from Avestin. The flow rate was set at between 15 to 20 litres/minute.

A batch of 100 ml of a 10% fenofibrate suspension was prepared in the following manner: 0.5% Vitamin E TPGS was preheated in a beaker to 40 degrees centigrade and this was added to water for injection. The mixture was stirred under magnetic stirring until a clear solution was formed. Fenofibrate was added under high shear, and the suspension was maintained under stirring until any foam disappeared. The particle size distribution was measured by laser diffraction

Suspension PSD determined by LD (Fraunhofer) need to be D50<15 μm and D99.9<50 μm.

This coarse suspension was subjected to a pre-homogenisation step for 10 minutes at 500b in order to standardise the particle population, before being homogenised for 60 minutes at 1500b.

The homogenisation process was carried out using four different valve designs, which are set forth in FIG. 4.

The results of the homogenisation process for each valve configuration are set forth below:

### Vale Configuration 1

After 30 min of homogenization 1500b

D50=0.9 μm, D99=2.1 μm, D100=10.1 μm by laser diffraction (LD). No photo-correlation spectroscopy (PCS) possible.

### Valve Configuration 2

After 30 min of homogenization 1500b

D50=0.7 μm, D99=1.8 μm, D100=8.1 μm by LD and no PCS

After 60 min of homogenization 1500b

D50=0.6 μm, D99=1.6 μm, D100=2.4 μm by LD and no PCS

### Valve Configuration 3

Angle of piston=30°, piston angle similar to valve seat angle

After 30 min of homogenization 1500b

D50=0.7 μm, D99=0.9 μm, D100=1.4 μm by LD and no PCS

After 60 min of homogenization 1500b

D50=0.6 μm, D99=0.8 μm, D100=1.0 μm by LD, 450 nm/PI=0.3 by PCS

Angle of piston=15°, piston angle<valve angle

After 30 min of homogenization 1500b

D50=0.6 μm, D99=0.8 μm, D100=1.0 μm by LD, PCS not performed

After 60 min of homogenization 1500b

D50=0.6 μm, D99=0.7 μm, D100=0.9 μm by LD, 400 nm/PI=0.3 by PCS

### Valve Configuration 4

After 30 min of homogenization 1500b

D50=0.5 μm, D99=0.6 μm, D100=0.8 μm by LD, 380 nm/PI=0.3 by PCS

After 60 min of homogenization 1500b

D50=0.4 μm, D99=0.5 μm, D100=0.7 μm by LD, 330 nm/PI=0.4 by PCS

### EXAMPLE 2

100 ml of a 10% perphenazine suspension containing 0.5% simulsol 1285 (PEG-60 castor oil) and water for injection was prepared and homogenised in a manner described in Example 1. The results are set forth below:

### Valve Configuration 4

After 30 min of homogenization 1500b

D50=0.5 μm, D99=0.6 μm, D100=0.7 μm by LD, 360 nm/PI=0.3 by PCS

1. A method of forming fine particles of a drug substance in a piston gap homogeniser, said homogeniser comprising:
a body having a flow passage through which is caused to flow a coarse suspension of drug substance particles in a carrier fluid,
a homogenisation valve disposed in the flow passage,
said valve comprising first and second homogenising members, each having a homogenising surface that opposes the other such that together the opposing surfaces define a gap that acts to restrict the flow of said coarse suspension and through which said suspension is expressed and discharged as a suspension containing fine particles of the drug substance, wherein at least one of the opposing homogenisation surfaces is an arcuate surface;
wherein the coarse suspension contains crystalline material, mixtures of crystalline material, or amorphous particles of a drug substance stabilized by a surfactant or combination of surfactants.

2. A method according to claim 1 wherein the first homogenising member is a valve seat and the second homogenising member is a piston.

3. A method according to claim 1 wherein the homogenising surface on the piston is arcuate.

4. A method according to claim 3 wherein the homogenising surface on the piston is spherical.

5. A method according to claim 2 wherein the piston comprises a ball having a spherical homogenising surface.

6. A method according to claim 5 wherein the ball is releasably fixed to the piston.

7. A method according to claim 5 wherein the ball is formed of ceramics material, ceramics coated steel, or tungsten carbide.

8. A method according to claim 2 wherein the valve seat is housed in a valve body and is stationary, and the piston is moveable into and out of contact with the valve body such that with the valve in a closed position the homogenising surface on the piston contacts the homogenising surface on the valve seat in sealed relationship and with the valve in the open position the piston and valve homogenising surfaces are in spaced apart relationship and together define a gap through which drug substance particles suspended in a carrier fluid are expressed and discharged as a fine suspension.

9. A method according to claim 2 wherein the valve seat and piston are rotationally symmetrical and axially aligned and a suspension is pumped through an axial bore in the valve seat under pressure and expressed radially outwards though the gap between the piston and valve seat.

10. A method according to claim 1 wherein the suspension comprises in addition to particles of a drug substance and a carrier fluid, one or more surfactants.

11. (canceled)

12. A fine suspension prepared by the method of claim 1.

13. A fine suspension according to claim 12 having a particle size distribution of about 200 nm (D50) to about 4.99 μm (D100).

14. A method of stabilising the fine suspension of claim 12 comprising the step of drying the suspension in a fluid bed drier.

15. A method of forming a pharmaceutical composition comprising the step of admixing the fine suspension of claim 12 with one or more pharmaceutically acceptable excipients.

16. A pharmaceutical composition comprising fine particles of a drug substance formed according to a method as defined in claim 1.
